(19) United States
(12) Patent Application Publication
(10) Pub. No.: US 2002/0159954 A1
(43) Pub. Date: Oct. 31, 2002

(54) Aerodynamically light vaccine for active pulmonary immunization

(76) Inventors: Parker Small, Gainesville, FL (US); Bradley S. Bender, Gainesville, FL (US)

Correspondence Address: VAN DYKE & ASSOCIATES, P.A., 1630 HILLCREST STREET, ORLANDO, FL, 32803, US

(21) Appl. No.: 10/005,374

(22) Filed: Dec. 3, 2001

Related U.S. Application Data
(60) Provisional application No. 60/250,795, filed on Dec. 1, 2000.

(51) Int. Cl. A61K 39/12; A61K 39/02; A61K 9/14

(52) U.S. Cl. 424/46; 424/199.1; 424/200.1

(57) ABSTRACT

Improved aerodynamically light particles for vaccine delivery to the pulmonary system, and methods for their synthesis and administration are provided. In a preferred embodiment, the aerodynamically light vaccine:) are made of a biodegradable material and have a tap density less than 0.4 g/ml and a mass mean diameter between 5 μm and 30 μm. The particles may be formed of biodegradable materials such as biodegradable polymers. For example, the particles may be formed of a functionalized polyester graft copolymer consisting of a linear .alpha.-hydroxy-acid polyester backbone having at least one amino acid group incorporated therein and it least one poly(amino acid) side chain extending from an amino acid group in the polyester backbone. In one embodiment, aerodynamically light vaccine particles having a large mean diameter, for example greater than 5 μm, can be used for enhanced delivery of a vaccine agent to the alveolar region of the lung. The aerodynamically light vaccine particles incorporating an immunizing agent may be effectively aerosolized for administration to the respiratory tract to permit systemic or local delivery of wide variety of immunizing agents.

### BACKGROUND OF THE INVENTION

1. Field of the Invention

The present invention relates generally to biodegradable particles of low density and large size for active immunization via the pulmonary system.

2. Background to the Invention

Biodegradable particles have been developed for the controlled-release and delivery of protein and peptide drugs. Langer, R., Science, 249: 1527-1533 (1990). Examples include the use of biodegradable particles for gene therapy (Mulligan, R. C. Science, 260: 926-932 (1993)) and for ‘single-shot’ immunization by vaccine delivery (Eldridge et al., Mol. Immunol., 28: 287-294 (1991)).

Aerosols for the delivery of therapeutic agents to the respiratory tract have been developed. Adjei, A. and Garren, J. Pharm. Res, 7,565-569 (1990); and Zanen, P. and Lamm, J. -W. J. Int. J. Pharm. 114, 111-115 (19˜)5). The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchioli which then lead to the ultimate respiratory zone, the alveoli, or deep lung. Gonda, I. “Aerosols for delivery of therapeutic and diagnostic agents to the respiratory tract,” in Critical Reviews in Therapeutic Drug Carrier Systems 6:273-313, 1990. The deep lung, or alveoli, are the primary target of inhaled therapeutic aerosols for systemic drug delivery.

Inhaled aerosols have been used for the treatment of local lung disorders including asthma and cystic fibrosis (Anderson et al., Am, Rev. Respir. Dis., 140: 1317-1324 (1989)) and have potential for the systemic delivery of peptides and proteins as well (Patton and Platz, Advanced Drug Delivery Reviews, 8:179-196 (1992)). However, pulmonary drug delivery strategies present many difficulties for the delivery of macromolecules; these include protein denaturation during aerosolization, excessive loss of inhaled drug in the oropharyngeal cavity (often exceeding 80%), poor control over the site of deposition, irreproducibility of therapeutic results owing to variations in breathing patterns, the often too-rapid absorption of drug potentially resulting in local toxic effects, and phagocytosis by lung macrophages.

Considerable attention has been devoted to the Idesign of therapeutic aerosol inhalers to improve the efficiency of inhalation therapies. Timsina et. al., Int. J. Pharm. 101, 1-13 (1995); and Tansey, I. p., Spray Technol. Market 4,26-29(1994). Attention has also been given to the design of dry powder aerosol surface texture, regarding particularly the need to avoid particle aggregation, a phenomenon which considerably diminishes the efficiency of inhalation therapies owing to particle aggregation. French, D. L., Edwards, D. A. and Niven, R. W., J. Aerosol Sci. 27, 769 783 (1996). Attention has not been given to the possibility of using large particle size (>5 .mu.m) as a means to improve aerosolization efficiency, despite the fact that in'traparticle adhesion diminishes with increasing particle size. French, D. L., Edwards, D. A. and Niven, R. W. J. Aerosol Sci. 27, 769 783 (1996). This is because particles of standard mass density (mass density near 1 g/cm.sup.3) and mean diameters >5 .mu.m are known to deposit excessively in the upper airways or the inhaler device. Heyder, J. et al., J.Aerosol Sci., 17: 811-825 (1986). For this reason, dry powder aerosols for inhalation therapy are generally produced with mean diameters primarily in the range of <5 .mu.m. Ganderton, D., J. Biopharmaceutical Sciences 3: 101-105 (1992); and Gonda, I. “Physico-Chemical Principles in Aerosol Delivery,” in Topics in Pharmaceutical Sciences 1991, Crommelin, D. J. and K. K. Midha, Eds., Medpharm Scientific Publishers, Stuttgart, pp. 95-115, 1992. Large “carrier” particles (containing no drug) have been co-delivered with therapeutic aerosols to aid in achieving efficient aerosolization among other possible benefits. French, D. L., Edwards, D. A. and Niven, R. W. J. Aerosol Sci. 27,769-783 (1996).

Local and systemic inhalation therapies can often benefit from a relatively slow controlled release of the therapeutic agent. Gonda, I., “Physico-chemical principles in aerosol delivery,” in: Topics in Pharmaceutical Sciences 1991, D. J. A. Crommelin and K. K. Midha, Eds., Stuttgart: Medpharm

Scientific Publishers, pp. 95-117, (1992). Slow release from a therapeutic aerosol can prolong the residence of an administered drug in the airways or acini, and diminish the rate of drug appearance in the bloodstream. Also, patient compliance is increased by reducing the frequency of dosing. Langer, R., Science, 249:1527-1533 (1990); and Gonda, I. “Aerosols for delivery of therapeutic and diagnostic agents to the respiratory tract,” in Critical Reviews in Therapeutic Drug Carrier Systems 6:273-313, (1990).

The human lungs can remove or rapidly degrade hydrolytically cleavable deposited aerosols over periods ranging from minutes to hours. In the upper airways, ciliated epithelia contribute to the “mucociliary escalator” by which particles are swept from the airways toward the mouth. Pavia, D. “Lung Mucociliary Clearance,” in Aerosols and the Lung: Clinical and Experimental Aspects, Clarke, S. W. and Pavia, D., Eds., Butterworths, London, 1984. Anderson et al., Am. Rev. Respir. Dis., 140: 1317-1324 (1989). In the deep lungs, alveolar macrophages; are capable of phagocytosing particles soon after their deposition. Warheit, M. B. and Hartsky, M. A., Microscopy Res. Tech. 26: 412-422 (1993); Brain, J. D., “Physiology and Pathophysiology of Pulmonary Macrophages,” in The Reticuloendothelial System, S. M. Reichard and J. Filkins, Eds., Plenum, New York, pp. 315-327, 1985; Dorries, A. M. and Valberg, P. A., Am. Rev. Resp. Disease 146, 831-837 (1991); and Gehr, P. et al. Microscopy Res. and Tech., 26, 423-436 (1993). As the diameter of particles exceeds 3 .mu.m, there is increasingly less phagocytosis by macrophages. Kawaguchi, H. (et al., Biomaterials 7: 61-66 (1986); Krenis, L. J. and Strauss, B., Proc. Soc. Exp. Med., 107:748 750 (1961); and Rudt, S. and Muller, R. H., J. Contr. Rel., 22: 263-272 (1992). However, increasing the particle size also minimizes the probability of particles (possessing standard mass density) entering the airways and acini due to excessive deposition in the oropharyngeal or nasal regions. Heyder, J. et al., J. Aerosol Sci., 17: 811-825 (1986). An effective dry-powder inhalation therapy for both short and long term release of therapeutics, either for local or systemic delivery, requires a powder that displays minimum aggregation, as well as a means of avoiding or suspending the lung's natural clearance mechanisms until drugs have been effectively delivered.

With respect to pulmonary delivery of drugs, U.S. Pat. Nos. 6,136,295; 5,985,309; 5,874,064; and 5,855,913 are hereby incorporated by reference for their disclosure of methods of deep lung delivery of agents other than vaccines. There remains, however, a need for improved inhaled aerosols for pulmonary delivery of vaccine agents. There is a need for the development of vaccine carriers and compositions, which are capable of delivering the vaccine in an effective amount into the airways or the alveolar zone of the lung. There further is a need for the development of vaccine carriers and compositions for use as inhaled aerosols, which are biodegradable and are capable of controlled release of vaccines within the airways or in the alveolar zone of the lung.

It is therefore an object of the present invention to provide improved carriers for the pulmonary delivery of vaccination agents.

It is a further object of the invention to provide inhaled aerosols which are effective carriers for delivery of vaccination agents to the deep lung.

It is another object of the invention to provide carriers for pulmonary delivery of vaccines which avoid phagocytosis in the deep lung.

It is a further object of the invention to provide carriers for pulmonary vaccine delivery which are capable of biodegrading and releasing the vaccine at a controlled rate. Further objects and advantages of this invention will be appreciated from a review of the complete disclosure.

### SUMMARY OF THE INVENTION

Improved aerodynamically light particles for vaccine delivery to the pulmonary system, and methods for their synthesis and administration are provided. In a preferred embodiment, the particles are made of a biodegradable material, have a tap density less than 0.4 g/cm.sup.3 and a mean diameter between 5 .mu.m and 30 .mu.m. In one embodiment, for example, at least 90% of the particles have a mean diameter between 5 mu.m and 30 .mu.m. The particles may be formed of biodegradable materials such as biodegradable polymers, proteins, or other water-soluble materials.

For example, the particles may be formed of a functionalized polyester graft copolymer consisting of a linear .alpha.-hydroxy-acid polyester backbone having at least one amino acid residue incorporated per molecule therein and at least one poly(amino acid) side chain extending from an amino acid group in the polyester backbone. Other examples include particles formed of water-soluble excipients, such as trehalose or lactose, or proteins. The aerodynamically light particles can be used for enhanced delivery of a vaccination agent to the airways or the alveolar region of the lung. The particles incorporating a vaccine agent may be effectively aerosolized for administration to the respiratory tract to permit systemic or local delivery of a wide variety of vaccine agents. They optionally may be co-delivered with larger carrier particles, not carrying a vaccinating agent, which have for example a mean diameter ranging between about 50 .mu.m and 100 .mu.m.

### DETAILED DESCRIPTION OF THE INVENTION

We disclose a method for producing small, light particles, containing vaccine antigens and delivery of vaccines via the respiratory tract: that protect against many diseases. In one embodiment, standard influenza vaccine that is usually administered by intramuscular injection is incorporated into low-density particles about 10 micrometers in diameter. This is inhaled by adults and cooperative children using a commercially available device developed by Alkermes.

The approach is applicable to other attenuated or inactivated virus vaccines including, but not limited to, diphtheria, tetanus, pertussus, polio and hepatitis A & B. The system is also applicable to administration of polysaccharide vaccines, such as pneumococcal polysaccharide vaccines and for polysaccharides linked to proteins such as the newer pneumococcal vaccines and HiB (haemopholis influenza B), and live virus vaccines, such as measles, mumps and rubella.

In a preferred embodiment, MV A vectored influenza vaccine is administered according to the method of this invention. This induces serum IgG and mucosal IgA antibody which prevents viral pneumonia and upper respiratory infection plus cell-mediated immunity which enhances recovery from flu infection including recovery from those viruses that may have drifted or shifted from those incorporated in the vaccine. Furthermore, genes from multiple pathogens are introduced into MVA so as to provide a multivalent, safe, effective vaccine that may not require refrigeration. Such a vaccine meets the requirements of the Children's Vaccine Initiative and is ideal for the developing world as well as the developed world.

The major practical advantage is that the vaccine can be administered by inhaling the fluffy powder and NOT BY A SHOT. It could eventually be made available OTC. Ultimately, if a Modified Vaccinia Ankara type vectored multivalent vaccine is proven to be efficacious, it should not require refrigeration, making it very useful in developing nations.

The benefit is vaccination without injection, thereby avoiding the pain but also, in economically deprived areas, avoiding dangers associated with diseases spread by multiple use of needles (hepatitis, AIDS, etc.). For influenza, it provides protection of both the upper and lower respiratory tract, whereas the current vaccine usually protects only the lung, thus the proposed vaccine is more effective in preventing spread of the disease.

In certain embodiments, inclusion of a noisemaker into devices for children and a mask for infants is contemplated and considered desirable.

The people of the world need vaccines. Adults need influenza vaccine annually and other vaccines periodically. Children need vaccines at different ages. The current influenza vaccine induces serum IgG antibody and prevents viral pneumonia, but frequently fails to protect against upper respiratory infection and spread. The present system for influenza induces serum IgG antibody and also induces IgA antibody in respiratory mucus and, thereby, protects both the lower and upper respiratory tracts from infection.

Measles vaccine is particularly advantageously administered by this system as it is predicted to be efficacious in the first six months of life, whereas the current vaccines cannot be effectively administered before about a year of age. This leaves up to six months vulnerability to infection. Measles vaccine delivery via the present system would greatly enhance the worldwide measles eradication program. In the U.S., approximately 100,000,000 doses of influenza vaccine are given/year. Children's vaccines are administered to approximately 5,000,000 children/year, some vaccines once and some three or four times/year. Demand is increasing due to the availability of vaccines for more diseases. An influenza vaccine given without a shot that prevents both upper and lower respiratory infection might meet the demand of 200,000,000 doses/year, especially if available OTC.

The following represent research done in humans approximately 30 years ago demonstrating the efficacy of using the respiratory route for immunization: Immunization Against Influenza, Waldman, R. H., Mann, J. J., Small, P. A. JAMA, 207, 520-524. 1969; An Evaluation of Influenza Immunization: Influence of Route of Administration and Vaccine Strain. Waldman, R. H. et al., Bulletin World Health Organization, 41,543-548, 1969. However, that work did not include the present improvement of efficient delivery of the vaccine to the alveoli. Use of particles for delivery of drugs to the alveoli is described in “Large Porous Particles for Pulmonary Drug Delivery”, by Edwards, D. A., Hanes, J. et al. Science, 276, 1868-1871, 1997. However, those authors did not disclose or suggest active immunization, as disclosed herein.

Focusing just on influenza, this vaccine would replace the existing inactivated flu vaccine and the live attenuated flu vaccine for adults. It is unclear whether the live attenuated or the proposed vaccine would be better for children who make up a small fraction of the market (probably <5%).

Aerodynamically light, biodegradable particles for improved delivery of vaccine agents to the respiratory tract are provided. The particles can be used in one embodiment for controlled systemic or local drug delivery to the respiratory tract via aerosolization. In a preferred embodiment, the particles have a tap density less than about 0.4 g/cm.sup.3. Features of the particle which can contribute to low tap density include irregular surface texture and porous structure. Administration of the low-density particles to the lung by aerosolization permits deep lung delivery of relatively large diameter immunizing aerosols, for example, greater than 5 .mu.m in mean diameter. A rough surface texture also can reduce particle agglomeration and provide a highly flowable powder, which is ideal for aerosolization via dry powder inhaler devices, leading to lower deposition in the mouth, throat and inhaler device.

Density and Size of Aerodynamically Light Particles

Particle Size

The mass mean diameter of the particles can be measured using a Coulter Counter. The aerodynamically light particles are preferably at least about 5 microns in diameter. The diameter of particles in a sample will range depending upon on factors such as particle composition and methods of synthesis. The distribution of size of particles in a sample can be selected to permit optimal deposition within targeted sites within the respiratory tract.

The aerodynamically light particles may be fabricated or separated, for example by filtration, to provide a particle sample with a preselected size distribution. For example, greater than 30%, 50%, 70%, or 80% of the particles in a sample can have a diameter within a selected range of at least 5 mu.m. The selected range within which a certain percentage of the particles must fall may be for example, between about 5 and 30 .mu.m, or optionally between 5 and 15 .mu.m. In one preferred embodiment, at least a portion of the particles have a diameter between about 9 and 11 .mu.m. Optionally, the particle sample also can be fabricated wherein at least 90%, or optionally 95% or 99%, have a diameter within the selected range. The presence of the higher proportion of the aerodynamically light, larger diameter (at least about 5 .mu.m) particles in the particle sample enhances the delivery of vaccinating agents incorporated therein to the deep lung.

In one embodiment, in the particle sample, the interquartile range may be 2 .mu.m, with a mean diameter for example of 7.5, 8.0, 8.5, 9.0, 9.5, 10.0, 10.5, 11.0, 11.5, 12.0, 12.5, 13.0 or 13.5 .mu.m. Thus, for example, at least :30%, 40%, 50% or 60% of the particles may have diameters within the selected range 5.5 7.5 .mu.m, 6.0-8.0 .mu.m, 6.5-8.5 .mu.m, 7.0-9.0 .mu.m, 7.5-9.5 .mu.m, 8.0-10.0 .mu.m, 8.5-10.5 .mu.m, 9.0-11.0 .mu.m, 9.5-11.5 .mu.m, 10.0-12.0 .mu.m, 10.5-12.5 .mu.m, 11.0-13.0 .mu.m, 11.5-13.5 .mu.m, 12.0-14.0 .mu.m, 12.5-14.5 .mu.m or 13.0-15.0 .mu.m. Preferably the said percentages of particles have diameters within a 1 .mu.m range, for example, 6.0 7.0 .mu.m, 10.0-11.0 mu.m or 13.0-14.0 .mu.m. The aerodynamically light particles incorporating a vaccine agent, and having a tap density less than about 0.4 g/cm.sup.3, with mean diameters of at least about 5 .mu.m, are more capable-of escaping inertial and gravitational deposition in the oropharyngeal region, and are targeted to the airways or the deep lung. The use of larger particles (mean diameter at least about 5 .mu.m) is advantageous since they are able to aerosolize more efficiently than smaller, non-light aerosol particles such as those currently used for inhalation therapies.

In comparison to smaller non-light particles, the larger (at least about 5 .mu.m) aerodynamically light particles also can potentially more successfully avoid phagocytic engulfment by alveolar macrophages and clearance from the lungs, due to size exclusion of the particles from the phagoeytes' cytosolic space. Phagocytosis of particles by alveolar macrophages diminishes precipitously as particle diameter increases beyond 3 mu.m. Kawaguchi, H. et al., Biomaterials 7: 61.66 (1986); Krenis, L. J. and Strauss, B., Proc. Soc. Exp. Med., 107:748-750 (1961); and Rudt, S. and Muller, R. H., J. Contr. Rei., 22: 263-272 (1992). For particles of statistically isotropic shape (on average, particles of the powder possess no distinguishable orientation), such as spheres with rough surfaces, the particle envelope volume is approximately equivalent to the volume of cytosolic space required within a macrophage for complete particle phagocytosis.

Aerodynamically light particles thus are capable of a longer-term release of a vaccinating agent. Following inhalation, aerodynamically light biodegradable particles can deposit in the lungs (due to their relatively low tap density), and subsequently undergo slow degradation and vaccine release, without the majority of the particles being phagocytosed by alveolar macrophages. The vaccine can be delivered relatively slowly into the alveolar fluid, and at a controlled rate into the blood stream, minimizing possible toxic responses of exposed cells to an excessively high concentration of the vaccine. The aerodynamically light particles thus are highly suitable for inhalation therapies, particularly in controlled release applications. The preferred mean diameter for aerodynamically light particles for inhalation therapy is at least about 5 .mu.m, for example between about 5 and 30 mu.m.

The particles may be fabricated with the appropriate material, surface roughness, diameter and tap density for localized delivery to selected regions of the respiratory tract such as the deep lung or upper airways. For example, higher density or larger particles may be used for upper airway delivery, or a mixture of different sized particles in a sample, provided with the same or different vaccine may be administered to target different regions of the lung in one administration.

Particle Density and Deposition

The particles having a diameter of at least about 5 .mu.m and incorporating a vaccine agent preferably are aerodynamically light. As used herein, the phrase “aerodynamically light particles” refers to particles having a tap density less than about 0.4 g/cm.sup.3. The tap density of particles of a dry powder may be obtained using a GeoPyc.TM. (Micrometrics Instrument Corp., Norcross, Ga. 30093). Tap density is a standard measure of the envelope mass density. The envelope mass density of an isotropic particle is defined as the mass of the particle divided by the minimum sphere envelope volume within which it can be enclosed.

Inertial impaction and gravitational settling of aerosols are predominant deposition mechanisms in the airways and acini of the lungs during normal breathing conditions. Edwards, D. A., J. Aerosol Sci. 26:293-317 (1995). The importance of both deposition mechanisms increases in proportion to the mass of aerosols and not to particle (or envelope) volume. Since the site of aerosol deposition in the lungs is determined by the mass of the aerosol (at least for particles of mean aerodynamic diameter greater than approximately 1. mu.m), diminishing the tap density by increasing particle surface irregularities and particle porosity permits the delivery of larger particle envelope volumes into the lungs, all other physical parameters being equal.

The low tap density particles have a small aerod,ynamic diameter in comparison to the actual envelope sphere diameter. The aerodynamic diameter, d.sub.aer, is related to the envelope sphere diameter, d (Gonda, I., “Physico-chemical principles in aerosol delivery,” in Topics in Pharmaceutical Sciences 1991 (Eds. D. J. A. Crommelin and K. K. Midha), pp. 95-117, Stuttgart: Medpharm Scientific Publishers, 1992) by the formula:

d.sub.aer=d.sqroot . . . rho.

where the envelope mass rho. is in units of g/cm.sup.3. Maximal deposition of monodisperse aerosol particles in the alveolar region of the human lung (about.60%) occurs for an aerodynamic diameter of approximately d.sub.aer=3 .mu.m. Heyder, J. et al., J. Aerosol Sci., 17: 811-825 (1986). Due to their small envelope mass density, the actual diameter d of aerodynamically light particles comprising a monodisperse inhaled powder that will exhibit maximum deep-lung deposition is:

d=3/.sqroot..rho..mu.m (where .rho.<1 g/cm.sup.3);

where d is always greater than 3 .mu.m. For example, aerodynamically light particles that display an envelope mass density, .rho.=0.1 g/cm.sup.3, will exhibit a maximum deposition for particles having envelope diameters as large as 9.5 .mu.m. The increased particle size diminishes interparticle adhesion forces. Visser, J., Powder Technology, 58: 1-10. Thus, large particle size increases efficiency of aerosolization to the deep lung for particles of low envelope mass density, in addition to contributing to lower phagocytic losses.

Particle Materials

In order to serve as efficient and safe vaccine carriers in vaccine delivery systems, the aerodynamically light particles preferably are biodegradable and biocompatible, and optionally are capable of biodegrading at a controlled rate for delivery of a vaccine. The particles can be made of any material which is capable of forming a particle having a tap density less than about 0.4 g/cm.sup.3. Both inorganic and organic materials can be used. For example, ceramics may be used. Other non-polymeric materials (e.g. fatty acids) may be used which are capable of forming aerodynamically light particles as defined herein. Different properties of the particle can contribute to the aerodynamic lightness including the composition forming the particle, and the presence of irregular surface structure or pores or cavities within the particle.

Polymeric Particles

The particles may be formed from any biocompatible, and preferably biodegradable polymer, copolymer, or blend, which is capable of forming particles having a tap density less than about 0.4 g/cm.sup.3.

Surface eroding polymers such as polyanhydrides may be used to form the aerodynamically light particles. For example, polyanhydrides such as poly[(p-carboxyphenoxy)-hexane anhydride] (PCPH) may be used. Biodegradable polyanhydrides are described, for example, in U.S. Pat. No. 4,857,311, the disclosure of which is incorporated herein by reference.

In another embodiment, bulk-eroding polymers such as those based on polyesters including poly(hydroxy acids) can be used. For example, polyglycolic acid (PGA) or polylactic acid (PLA) or copolymers thereof may be used to form the aerodynamically light particles, wherein the polyester has incorporated therein a charged or functionalizable group such as an amino acid as described below.

Other polymers include polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl ethers, and polyvinyl esters, polymers of acrylic and methacrylic acids, celluloses and other polysaccharides, and peptides or proteins, or copolymers or blends thereof which are capable of forming aerodynamically light particles with a tap density less than about 0.4 g/cm.sup.3. Polymers may be selected with or modified to have the appropriate stability and degradation rates in vivo for different controlled vaccine delivery applications.

Polyester Graft Copolymers

In one preferred embodiment, the aerodynamically light particles are formed from functionalized polyester graft copolymers, as described in Hrkach et al., Macromolecules, 28:4736-4739 (1995); and Hrkach et at., “Poly(L-Lactic acid-co-amino acid) Graft Copolymers: A Class of Functional Biodegradable Biomaterials” in Hydrogels and Biodegradable Polymers for Bioapplications, ACS Symposium Series No.627, Raphael M. Ottenbrite et al., Eds., American Chemical Society, Chapter 8, pp. 93-101, 1996, the disclosures of which are incorporated herein by reference. The functionalized graft copolymers are copolymers of polyesters, such as poly(glycolic acid) or poly(lactic acid), and another polymer including functionalizable or ionizable groups, such as a poly(aminoacid). In a preferred embodiment, comb-like graft copolymers are used which include a linear polyester backbone having amino acids incorporated therein, and poly(amino acid) side chains which extend from the amino acid residues in the polyester backbone. The polyesters may be polymers of alpha-hydroxy acids such as lactic acid, glycolic acid, hydroxybutyric acid and hydroxy valeric acid, or derivatives or combinations thereof. The inclusion of ionizable side chains, such as polylysine, in the polymer has been found to enable the formation of more aerodynamically light particles, using techniques for making microparticles known in the art, such as solvent evaporation. Other ionizable groups, such as amino or carboxyl groups, may be incorporated, covalently or noncovalently, into the polymer to enhance surface roughness and porosity. For example, polyalanine could be incorporated into the polymer.

An exemplary polyester graft copolymer, which may be used to form aerodynamically light polymeric particles is the graft copolymer, poly(lactic acid-co-lysine-graft-lysine) (PLAL-Lys), which has a polyester backbone consisting of poly(L-lactic acid-co-L-lysine) (PLAL), and grafted poly-lysine chains. PLAL-Lys is a comb-like graft copolymer having a backbone composition, for example, of 98 mol % lactic acid and 2 mol % lysine and poly(lysine) side chains extending from the lysine sites of the backbone.

PLAL-Lys may be synthesized as follows. First, the PLAL copolymer consisting of L-lactic acid units and approximately 1-2% N epsilon, carbobenzoxy-L-lysine (Z-L-lysine) units is synthesized as described in Barrera et al., J. Am. Chem. Soc.,

115:11010 (1993). Removal of the Z protecting groups of the randomly incorporated lysine groups in the polymer chain of PLAL yields the free .epsilon.-amine which can undergo further chemical modification. The use of the poly(lactic acid) copolymer is advantageous since it biodegrades into lactic acid and lysine, which can be processed by the body. The existing backbone lysine groups are used as initiating sites for the growth of poly(amino acid) side chains.

The lysine epsilon-amino groups of linear poly(L-lactic acid-co-L-lysine) copolymers initiate the ring opening polymerization of an amino acid N-.epsilon. carboxyanhydride (NCA) to produce poly(L-lactic acid-co-amino acid) comb-like graft copolymers. In a preferred embodiment, NCAs are synthesized by reacting the appropriate amino acid with triphosgene. Daly et al., Tetrahedron Lett., 29:5859 (1988). The advantage of using triphosgene over phosgene gas is that it is a solid material, and therefore, safer and easier to handle. It also is soluble in THF and hexane so any excess is efficiently separated from the NCAs.

The ring opening polymerization of amino acid N-carboxyanhydrides (NCAs) is initiated by nucleophilic initiators such as amines, alcohols, and water. The primary amine initiated ring opening polymerization of NCAs allows good control over the degree of polymerization when the monomer to initiator ratio (M/I) is less than 150. Kricheldorf, H. R. in Models of Biopolymers by Ring Opening Polymerization, Penczek, S., Ed., CRC Press, Boca Raton, 1990, Chapter 1; Kricheldorf, H. R. .alpha.-Aminoacid-N-Carboxy-Anhydrides and Related Heterocycles, Springer-Verlag, Berlin, 1987; and Imanishi, Y. in Ring-Opening Polymerization, Ivin, K. J. and Saegusa, T., Eds., Elsevier,

London, 1984, Volume 2, Chapter 8. Methods for using lysinc epsilon, amino groups as polymeric initiators for NCA polymerizations are describe,d in the art. Sela, M. et al., J. Am. Chem. Soc., 78: 746 (1956).

In the reaction of an amino acid NCA with PLAL, the nucleophilic primary epsilon.amino of the lysine side chain attacks C-5 of the NCA leading to ring opening and formation of the amino acid amide, along with the evolution ofCO.sub.2. Propagation takes place via further attack of the amino group of the amino acid amides on subsequent NCA molecules. The degree of polymerization of the poly(amino acid) side chains, the corresponding amino acid content in the graft copolymers and their resulting physical and chemical characteristics can be controlled by changing the M/I ratio for the NCA polymerization—that is, changing the ratio of NCA to lysine .epsilon.-amino groups of pLAL. Thus, in the synthesis, the length of the poly(amino acid), such as poly(lysine), side chains and the total amino acid content in the polymer may be designed and synthesized for a particular application.

The poly(amino acid) side chains grafted onto or incorporated into the polyester backbone can include any amino acid, such as aspartic acid, alanine or lysine, or mixtures thereof. The functional groups present in the amino acid side chains, which can be chemically modified, include amino, carboxylic acid, thiol, guanido, imidazole and hydroxyl groups. As used herein, the term “amino acid” includes natural and synthetic amino acids and derivatives thereof. The polymers can be prepared with a range of amino acid side chain lengths, for example, about 10-100 or more amino acids, and with an overall amino acid content of, for example, 7-72% or more depending on the reaction conditions. The grafting of poly(amino acids) from the pLAL backbone may be conducted in a solvent such as dioxane, DMF, or CH.sub.2 CI.sub.2, or mixtures thereof. In a preferred embodiment, the reaction is conducted at room temperature for about 2-4 days in dioxane.

Alternatively, the aerodynamically light particles for pulmonary vaccine delivery may be formed from polymers or blends of polymers with different polyester/amino acid backbones and grafted amino acid side chains. For example, poly(lactic acid-co-lysine-graft-alanine-lysine) (PLAL-Ala-Lys), or a blend ofPLAL-Lys with poly(lactic acid-co-glycolic acid-block-ethylene oxide) (PLGA-PEIG) (PLAL-Lys-PLGA-PEG) may be used.

In the synthesis, the graft copolymers may be tailored to optimize different characteristics of the aerodynamically light particle including: i) interactions between the agent to be delivered and the copolymer to provide stabilization of the agent and retention of activity upon delivery; ii) rate of polymer degradation and, thereby, rate of vaccine release profiles; iii) surface characteristics and targeting capabilities via chemical modification; and iv) particle porosity.

Formation of Aerodynamically Light Polymeric Particles

Aerodynamically light polymeric particles may be prepared using single and double emulsion solvent evaporation, spray drying, solvent extraction and other methods well known to those of ordinary skill in the art. The aerodynamically light particles may be made, for example using methods for making microspheres or microcapsules known in the art.

Methods developed for making microspheres for drug delivery are described in the literature, for example, as described by Mathiowitz and Langer, J. Controlled Release 5,13-22 (1987); Mathiowitz, et al., Reactive Polymers 6,275-283 (1987); and Mathiowitz, et al., J. Appl. Polymer Sci. 35,755-774 (1988), the teachings of which are incorporated herein. The selection of the method depends on the polymer selection, the size, external morphology, and crystallinity that is desired, as described, for example, by Mathiowitz, et al., Scanning Microscopy 4,329-340 (1990); Mathiowitz, et al., J. Appl. Polymer Sci. 45, 125-134 (1992); and Benita, el: al., J. Pharm. Sci. 73, 1721-1724 (1984), the teachings of which are incorporated herein.

In solvent evaporation, described for example, in Mathiowitz, et al., (1990), Benita, and U.S. Pat. No. 4,272,398 to Jaffe, the polymer is dissolved in a volatile organic solvent, such as methylene chloride. Several different polymer concentrations can be used, for example, between 0.05 and 0.20 g/ml. The drug, either in soluble form or dispersed as fine particles, is added to the polymer solution, and the mixture is suspended in an aqueous phase that contains a surface-active agent such as poly(vinyl alcohol). The aqueous phase may be, for example, a concentration of 1% poly(vinyl alcohol) w/v in distilled water. The resulting emulsion is stirred until most of the organic solvent evaporates, leaving solid microspheres, which may be washed with water and dried overnight in a lyophilizer.

Microspheres with different sizes (1-1000 microns) and morphologies can be obtained by this method which is useful for relatively stable polymers such as polyesters and polystyrene. However, labile polymers such as polyanhydrides may degrade due to exposure to water. For these polymers, solvent removal may be preferred.

Solvent removal is primarily designed for use with polyanhydrides. In this method, the drug is dispersed or dissolved in a solution of a selected polymer in a volatile organic solvent like methylene chloride. The mixture is then suspended in oil, such as silicon oil, by stirring, to form an emulsion. Within 24 hours, the solvent diffuses into the oil phase and the emulsion droplets harden into solid polymer microspheres. Unlike solvent evaporation, this method can be used to make microspheres from polymers with high melting points and a wide range of molecular weights. Microspheres having a diameter for example between one and 300 microns can be obtained with this procedure.

Targeting of Particles

Targeting molecules can be attached to the aerodynamically light particles via reactive functional groups on the particles. For example, targeting molecules can be attached to the amino acid groups of functionalized polyester graft copolymer particles, such as PLAL-Lys particles. Targeting molecules permit binding interaction of the particle with specific receptor sites, such as those within the lungs. The particles can be targeted by attachment of ligands which specifically or non.-specifically bind to particular targets. Exemplary targeting molecules include antibodies and fragments thereof including the variable regions, lectins, and hormones or other organic molecules capable of specific binding for example to receptors on the surfaces of the target cells.

Vaccine Agents

The aerodynamically light polymeric aerosols are useful as carriers for a variety of vaccine agents, including but not limited to recombinant viral vaccines, such as recombinant modified vaccinia vaccine incorporating influenza virus antigens, killed virus, attenuated virus vaccines, bacterial vaccines, including attenuated bacterial pathogens, inactivated bacterial pathogens, and recombinant bacteria expressing specific antigens against which elicitation of an immune: response is desired. They can be used to encapsulate small and large viral or bacterial antigens, release encapsulated vaccines over time periods ranging from hours to months, and withstand extreme conditions during aerosolization or following deposition in the lungs that might otherwise harm the encapsulated vaccine. Attention to disruption of the vaccine is required in the formation of the vaccine -carrier composition. Exposure to detergents, excesses of heat or organic solvents are to be avoided, where the vaccine entity is susceptible to disruption to these agents. HIV, hepatitis, herpes, or any other viral or bacterial disease may be prevented by administering an appropriate vaccine according to this invention.

Administration

The particles including a vaccine agent may be administered alone or in any appropriate pharmaceutical carrier, such as a liquid, for example saline, or a powder, for administration to the =respiratory system. They can be co-delivered with larger carrier particles, not including a vaccine agent, the latter possessing mass mean diameters for example in the range 50 .mu.m-100 .mu.m.

Aerosol dosage, formulations and delivery systems may be selected for a particular vaccine application, as described, for example, in Gonda, I. “Aerosols for delivery of therapeutic and diagnostic agents to the respiratory tract,” in Critical Reviews in Therapeutic Drug Carrier Systems, 6:273-313, 1990; and in Moren, “Aerosol dosage forms and formulations,” in: Aerosols in Medicine. Principles, Diagnosis and Therapy, Moren, et al., Eds, Esevier, Amsterdam, 1985, the disclosures of which are incorporated herein by reference. Typically, dosages of vaccines which correspond to those in use for systemic delivery by injection should be delivered by the pulmonary route described herein.

The greater efficiency of aerosolization by aerodynamically light particles of relatively large size permits more vaccine to be delivered than is possible with the same mass of non-light aerosols. The relatively large size of aerodynamically light aerosols depositing in the deep lungs also minimizes potential vaccine losses caused by particle phagocytosis. The use of aerodynamically light polymeric aerosols as therapeutic carriers provides the benefits of biodegradable polymers for controlled release in the lungs and long-time local action or systemic bioavailability. Denaturation of vaccines can be minimized during aerosolization since the vaccine agents are contained and protected within a polymeric shell. Coencapsulation of peptides with peptidase-inhibitors can minimize enzymatic degradation of key antigenic determinants of the vaccine.

The present invention will be further understood by reference to the following non-limiting examples.

### EXAMPLE 1

### Synthesis of Aerodynamically Light Poly[(p-carboxyphenoxy)-hexane anhydride] (“PCPH”) Particles

Aerodynamically light poly[(p-carboxyphenoxy)-hexane anhydride] (“PCPH”) particles are synthesized as follows. 100 mg PCPH (MW .about.25,000) is dissolved in 3.0 mL methylene chloride. To this clear solution is added 5.0 mL 1% w/v aqueous polyvinyl alcohol (PVA, MW .about.25,000, 88 mole % hydrolyzed) saturated with methylene chloride, and the mixture is vortexed (Vortex Genie 2, Fisher Scientific) at maximum speed for one minute. The resulting milky-white emulsion is poured into a beaker containing 95 mL 1% PV A and homogenized (Silverson Homogenizers) at 6000 RPM for one minute using a 0.75 inch tip. After homogenization, the mixture is stirred with a magnetic stirring bar and the methylene chloride quickly extracted from the polymer particles by adding 2 mL isopropyl alcohol. The mixture is continued to stir for 35 minutes to allow complete hardening of the microparticles. The hardened particles are collected by centrifugation and washed several times with double distilled water. The particles are freeze-dried to obtain a free-flowing powder void of clumps.

The mean diameter of this batch is 6.0 .mu.m, however, particles with mean diameters ranging from a few hundred nanometers to several millimetres may be made with only slight modifications. Scanning electron micrograph photos of a typical batch of PCPH particles showed the particles to be highly porous with irregular surface shape. The particles have a tap density less than 0.4 g/cm.sup.3.

### EXAMPLE 2

### Synthesis ofPLAL-Lys and PLAL-Lys-Ala Polymeric and Copolymeric Particles

### Aerodynamically Light PLAL-Lys Particles

PLAL-Lys particles are prepared by dissolving 50 mg of the graft copolymer in 0.5 ml dimethylsulfoxide, then adding 1.5 ml dichlorornethane dropwise. The polymer solution is emulsified in 100 ml of 5% w/v polyvinyl alcohol solution (average molecular weight 25 KDa, 88% hydrolyzed) using a homogenizer (Silverson) at a speed of approximately 7500 rpm. The resulting dispersion is stirred using a magnetic stirrer for 1 hour.

Following this period, the pH is brought to 7.0 7.2 by addition of 0.1 N NaOH solution. Stirring is continued for an additional 2 hours until the methylene chloride is completely evaporated and the particles hardened. The particles are then isolated by centrifugation at 4000 rpm (600 g) for 10 minutes (Sorvall RC-5B). The supernatant is discarded and the precipitate washed three times with distilled water followed by centrifugation for 10 minutes at 4000 rpm each time. Finally, the particles are resuspended in 5 ml of distilled water, the dispersion frozen in liquid nitrogen, and lyophilized (Labconco freeze dryer 8) for at least 48 hours. Particle sizing is performed using a Coulter counter. Average particle mean diameters ranged from 100 nm to 14 .mu.m, depending upon processing parameters such as homogenization speed and time. All particles exhibited tap densities less than 0.4 g/cm.sup.3. Scanning electron micrograph photos of the particles showed them to be highly porous with irregular surfaces.

### Aerodynamically Light PLAL-Ala-Lys Particle:5

100 mg of PLAL-Ala-Lys is completely dissolved in 0.4 ml trifluoroethanol, then 1.0 ml methylene chloride is added dropwise. The polymer solution is emulsified in 100 ml of 1% w/v polyvinyl alcohol solution (average molecular weight 25 KDa, 80% hydrolyzed) using a sonicator (Sonic & Materal VC-250) for 15 seconds at an output of 40 W. 2 ml of 1% PV A solution is added to the mixture and it is vortexed at the highest speed for 30 seconds. The mixture is quickly poured into a beaker containing 100 ml 0.3% PV A solution, and stirred for three hours allowing evaporation of the methylene chloride. Scanning electron micrograph photos of the particles showed them to possess highly irregular surfaces.

### Aerodynamically Light Copolymer Particles

Polymeric aerodynamically light particles consisting of a blend of PLAL-Lys and PLGA-PEG are made. 50 mg of the PLGA-PEG polynler (molecular weight of PEG: 20 KDa, 1:2 weight ratio of PEG:PLGA, 75:25 lactide:glycolide) is completely dissolved in 1 ml dichloromethane. 3 mg of poly(lactide-co-lysine)-polylysine graft copolymer is dissolved in 0.1 ml dimethylsulfoxide and mixed with the first polymer solution. 0.2 ml of TE buffer, pH 7.6, is emulsified in the polymer solution by probe sonication (Sonic &Materal VC-250) for 10 seconds at an output of40 W. To this first emulsion, 2 ml ofdistilled water is added and mixed using a vortex mixer at 4000 rpm for 60 seconds. The resulting dispersion is agitated by using a magnetic stirrer for 3 hours until methylene chloride is completely evaporated and microspheres formed. The spheres are then isolated by centrifugation at 5000 rpm for 30 min. The supernatant is discarded, the precipitate washed three times with distilled water and resuspended in 5 ml of water. The dispersion is frozen in liquid nitrogen and lyophilized for 48 hours.

Variables which may be manipulated to alter the size distribution of the particles include: polymer concentration, polymer molecular weight, surfactant type (e.g., PVA, PEG, etc.), surfactant concentration, and mixing intensity. Variables which may be manipulated to alter the surface shape and porosity of the particles include: polymer concentration, polymer molecular weight, rate of methylene chloride extraction by isopropyl alcohol (or another miscible solvent), volume of isopropyl alcohol added, inclusion of an inner water phase, volume of inner water phase, inclusion of salts or other highly water-soluble molecules in the inner water phase which leak out of the hardening sphere by osmotic pressure, causing the formation of channels, or pores, in proportion to their concentration, and surfactant type and concentration.

By scanning electron microscopy (SEM), the PLAL-Lys-PLGA-PEG particles are highly surface rough and porous. The particles had a mean particle diameter of 7 .mu.m. The blend of PLAL-Lys with poly(lactic acid) (PLA) and/or PLGA-PEG copolymers can be adjusted to adjust particle porosity and size. Additionally, processing parameters such as homogenization speed and time can be adjusted. Neither PLAL, PLA nor PLGA-PEG alone yields an aerodynamically light structure when prepared by these techniques.

### EXAMPLE 3

### Synthesis of Spray-Dried Particles

### Aerodynamically Light Particles Containing Polymer and Vaccine Soluble in Common Solvent

Aerodynamically light 50:50 PLGA particles are prepared by spray drying with vaccine encapsulated within the particles according to the following procedures. poly (D,L-lactic-co-glycolic acid) with a molar ratio of 50:50 (PLGA 50:50, Resomer RG503, B.I. Chemicals, Montvale, N.J.) and vaccine are completely dissolved in water or dichloromethane at room temperature. The mixture is subsequently spray-dried through a 0.5 mm nozzle at a flow rate of 5 mL/min using a Buchi laboratory spray-drier (model 190, Buchi, Germany). The flow rate of compressed air is 700 nl. The inlet temperature is set to 30.degree. C. and the outlet temperature to 25.degree. C. The aspirator is set to achieve a vacuum of −20 to −25 bar. The mean particle size is approximately 5 .mu.m. Larger particle size can be achieved by lowering the inlet compressed air flow rate, as well as by changing other variables. The particles are aerodynamically light, as determined by a tap density less than or equal to 0.4 g/cm.sup.3. Porosity and surface roughness can be increased by varying the inlet and outlet temperatures, among other factors.

### Aerodynamically Light Particles Containing Polymer and Vaccine in Different Solvents

Aerodynamically light PLA particles with a vaccine agent, recombinant MV A encoding influenza virus antigens, is prepared by spray drying using the following procedure. 2.0 mL of an aqueous vaccine solution is emulsified into 100 mL of a 2% w/v solution of poly (D,L-lactic acid) (PLA, Resomer R206, B.I. Chemicals) in dichloromethane by probe sonication (Vibracell Sonicator, Branson). The emulsion is subsequently spray-dried at a flow rate of 5 mL/min with an air flow rate of 700 nl/h (inlet temperature=30.degree. C., outlet temperature=21.degree. C., −20 mbar vacuum). The particles are aerodynamically light, as determined by a tap density less 0.4 g/cm.sup.3.

### Aerodynamically Light Vaccine Particles

Aerodynamically light vaccine particles are prepared by spray drying using the following procedure. An immunologically effective amount of an attenuated, inactivated or non-pathogenic recombinant viral or bacterial vaccine is dissolved in double distilled water or saline and spray-dried using a 0.5 mm nozzle and a Buchi laboratory spray-drier. The flow rate of compressed air is about 725 nl/h.

The flow rate of the vaccine solution is set such that, at a set inlet temperature of 97-100.de'gree. C., the outlet temperature is 55-57.degree. C. The aspirator is set to achieve a vacuum of −30 mbar. The immunogenic activity of the vaccine is found to be unaffected by this process.

### Aerodynamically Light Vaccine Particles

Aerodynamically light vaccine particles are prepared by spray drying using the following procedure. An immunologically effective amount of vaccine is dissolved in double distilled water or saline and spray-dried using a 0.5 mm nozzle and a Buchi laboratory spray-drier. The flow rate of compressed air is 750 nl/h. The flow rate of the vaccine solution is set such that, at a set inlet temperature of 155.degree. C., the outlet temperature is 80.degree. C. The aspirator is set to achieve a vacuum of−20 mbar.

### EXAMPLE 4

### In Vivo Aerosolization of PLAL and PLAL-Lys Vaccine Particles

The penetration of aerodynamically light and non-light polymeric PLAL-Lys and PLAL vaccine microparticles into the lungs is evaluated in an in vivo experiment involving the aerosolization of the microparticles into the airways of live rats.

Male Spraque Dawley rats (150-200 g) are anesthetized using ketamine (90 mg/kg)/xylazine (10 mg/kg). The anesthetized rat is placed ventral side up on a surgical table provided with a temperature-controlled pad to maintain physiological temperature. The animal is cannulated above the carina with an endotracheal tube connected to a Harvard ventilator. The animal is force ventilated for 20 minutes at 300 ml/min. 50 mg of aerodynamically light (PLAL-Lys) or non-light (PLA) microparticles including vaccine is introduced into the endotracheal tube.

Following the period of forced ventilation, the animal is permitted to develop IgG, IgA, and cellular immune responses over a period of days to several weeks. ELISA assays of pre-innoculation and post-innoculation time points are conducted on appropriate test antigens to demonstrate the elicitation of appropriate humoral immune responses, while standard cellular immune response assays are conducted to test elicitation of this component of the immune response.

Modifications and variations of the present invention will be obvious to those skilled in the art from the foregoing detailed description. Such modifications and variations are intended to come within the scope of the following claims.

What is claimed is:

1. Biocompatible particles for delivery of a vaccine to the pulmonary system comprising an immunizing agent; wherein the particles have a tap density less than 0.4 g/ml and at least 90% of the particles have geometric dimensions between about 5 μm and about 30 μm.

2. The particles of claim 1 wherein the immunizing agent is selected from the group consisting of a live attenuated virus or bacterial vaccine, a recombinant virus or bacterial vaccine encoding an immunizing antigen or a combination of antigens against which elicitation of an immune response is desired, and an inactivated virus or bacterial vaccine.

3. The particles of claim 1 combined with large biodegradable carrier particles having a mass mean diameter in the range of about 50 .mu.m to about 100 .mu.m.

4. The particles of claim 1 combined with a pharmaceutically acceptable carrier for administration to the respiratory tract.

5. The particles of claim 1 wherein at least 90% of the particles have a mass mean diameter between about 5 .mu.m and about 15 .mu.m.

6. The particles of claim 1 wherein at least 90% of the particles have a mean diameter between about 9 .mu.m and about 11 .mu.m.

7. The particles of claim 1 wherein at least 50% of the particles have a tap density of less than 0.1 g/cm.sup.3.

8. The particles of claim 1 wherein the particles further comprise a polymeric material.

9. The particles of claim 1 wherein the particles further comprise a non-polymeric material.

10. Biocompatible particles for delivery of a targeting molecule to the pulmonary system wherein the targeting molecule is attached to the particles and wherein the particles have a tap density less than 0.4 g/cm.sup.3, and at least 90% of the particles have geometric dimensions between 5 .mu.m and about 30 .mu.m.

11. Biocompatible particles for delivery of a vaccine agent to the pulmonary system comprising an immunologically effective amount of a vaccine agent; wherein the particles have a tap density less than 0.4 g/cm.sup.3 and at least 90% of the particles have an aerodynamic diameter between about 1 .mu.m and about 5 .mu.m.

12. The particles of claim 11 wherein the agent is selected from the group consisting of viral vaccines, bacterial vaccines, live, attenuated, recombinant, inactivated, and combinations thereof.

13. The particles of claim 11 combined with large biodegradable carrier particles having a mass mean diameter in the range of about 50 .mu.m to about 100 .mu.m.

14. The particles of claim 11 combined with a pharmaceutically acceptable carrier for administration to the respiratory tract.

15. The particles of claim 11 wherein at least 90% of the particles have an aerodynamic diameter between about 1 .mu.m and about 3 .mu.m.

16. The particles of claim 11 wherein at least 90% of the particles have an aerodynamic diameter between about 3 .mu.m and about 5 .mu.m.

17. The particles of claim 11 wherein at least 50% of the particles have a tap density of less than 0.1 g/cm.sup.3.

18. The particles of claim 11 wherein the particles further comprise a polymeric material.

19. The particles of claim 11 wherein the particles further comprise a non-polymeric material.

20. Biocompatible particles for delivery of a vaccine and targeting molecule to the pulmonary system wherein the targeting molecule is attached to the particles and wherein the particles have a tap density less than 0.4 g/cm.sup.3, and at least 90% of the particles have an aerodynamic diameter between about 1 .mu.m and about 5 .mu.m.

21. A method for delivery of an actively immunizing amount of a vaccine to the pulmonary system comprising: administering to the respiratory tract of a patient in need thereof of an effective amount of biocompatible particles incorporating said vaccine, wherein the particles have a tap density of less than about 0.4 g/cm.sup.3 and at least 90% of the particles have geometric dimensions between about 5 .mu.m and about 30 .mu.m.

22. The method of claim 21 wherein the agent is selected from the group consisting of viral vaccines, bacterial vaccines, live, attenuated, recombinant, inactivated, and combinations thereof.

23. The method of claim 21 wherein the particles are combined with large biodegradable carrier particles having a mass mean diameter in the range of about 50 .mu.m to about 100 .mu.m.

24. The method of claim 21 wherein the particles are combined with a pharmaceutically acceptable carrier for administration to the respiratory tract.

25. The method of claim 21 wherein at least 90% of the particles have a mass mean diameter between about 5 .mu.m and about 15 .mu.m.

26. The method of claim 21 for delivery to the alveolar zone of the lung wherein at least 90% of the particles have a mean diameter between about 9 and about 11 .mu.m.

27. The method of claim 21 wherein at least 50% of the administered particles have a tap density of less than about 0.1 g/cm.sup.3.

28. The method of claim 21 wherein the particles further comprise a polymeric material.

29. The method of claim 21 wherein the particles further comprise a non-polymeric material.

30. A method for delivery of a vaccine and a targeting molecule to the pulmonary system comprising: administering to the respiratory tract of a patient in need of treatment, prophylaxis or diagnosis an effective amount of biocompatible particles, wherein the particles have a tap density less than about 0.4 g/cm.sup.3 and at least 90% of the particles have geometric dimensions between about 5 .mu.m and about 30 .mu.m, and wherein the targeting molecule is attached to the particles which further comprise the vaccine.

31. A method for delivery of a vaccine to the pulmonary system comprising:
administering to the respiratory tract of a patient in need thereof of an effective amount of biocompatible particles comprising said vaccine, wherein the particles have a tap density of less than about 0.4 g/cm.sup.3 and at least 90% of the particles have an aerodynamic diameter between about 1 .mu.m and about 5 .mu.m.

32. The method of claim 31 wherein the agent is selected from the group consisting of viral vaccines, bacterial vaccines, live, attenuated, recombinant, inactivated, and combinations thereof.

33. The method of claim 31 wherein the particles are combined with large biodegradable carrier particles having a mass mean diameter in the range of about 50 .mu.m to about 100 .mu.m.

34. The method of claim 31 wherein the particles are combined with a pharmaceutically acceptable carrier for administration to the respiratory tract.

35. The method of claim 31 wherein at least 90% of the particles have an aerodynamic diameter between about 1 .mu.m and about 3 .mu.m.

36. The method of claim 31 for delivery to the alveolar zone of the lung wherein at least 90% of the particles have an aerodynamic diameter between about 3 .mu.m and about 5 .mu.m.

37. The method of claim 31 wherein at least 50% of the administered particles have a tap density of less than about 0.1 g/cm.sup.3.

38. The method of claim 31 wherein the particles further comprise a polymeric material.

39. The method of claim 31 wherein the particles further comprise a non-polymeric material.

40. A method for delivery of a vaccine and a targeting molecule to the pulmonary system comprising: administering to the respiratory tract of a patient in need of treatment, prophylaxis or diagnosis an effective amount of biocompatible particles comprising said vaccine, wherein the particles have a tap density less than about 0.4 g/cm.sup.3 and at least 90% of the particles have an aerodynamic diameter between about 1 .mu.m and about 5 .mu.m, and wherein the targeting molecule is attached to the particles.
